(19) United States
(12) Patent Application Publication
(10) Pub. No.: US 2008/0078404 A1
(43) Pub. Date: Apr. 3, 2008

(54) Endotracheal cuff and technique for using the same

(75) Inventor: Paul W. Martens, Pleasanton, CA (US)

Correspondence Address: Covidien;IP Counsel - Respiratory & Monitoring Solutions, 60 Middletown Avenue, North Haven, CT, 06473, US

(73) Assignee: Nellcor Puritan Bennett Incorporated

(21) Appl. No.: 11/541,194

(22) Filed: Sep. 29, 2006

(51) Int. Cl. A61M 16/00

(52) U.S. Cl. 128/207.15

(57) ABSTRACT

An inflatable balloon cuff may be adapted to include a gas barrier material in order to seal a patient's trachea when associated with an endotracheal tube. The gas barrier material may preserve a cuff's mechanical pressure seal during lengthy intubation times or during surgical procedures. The gas barrier on the inflatable cuff may be a general gas barrier, or may be specific to certain gases.

### BACKGROUND OF THE INVENTION

1. Field of the Invention

The present invention relates to medical devices, and more particularly, to endotracheal devices, such as endotracheal tubes and cuffs.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into the patient. For example, medical devices, such as tracheal tubes, may be used to control the flow of one or more substances into or out of a patient. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

Tracheal tubes may be used to control the flow of air or other gases through a patient's trachea. Such tracheal tubes may include endotracheal (ET) tubes, or tracheotomy tubes. To seal these types of tracheal tubes, an inflatable cuff may be associated with these tubes. When inflated, the cuff generally expands into the surrounding trachea to seal the tracheal passage around the circumference of the tube. A high-quality seal against the tracheal passageway allows a patient ventilator to perform efficiently.

As many patients are intubated for several days, healthcare workers may need to balance achieving a high-quality tracheal seal with possible patient discomfort. For example, the pressure of an inflated cuff against the tracheal walls may result in some discomfort for a patient. While a cuff may be inflated at lower pressure to avoid this discomfort, this may lower the quality of the cuff's seal against the trachea. Low cuff inflation pressures may also be associated with allowing folds to form in the walls of the cuff that may serve as leak paths for air as well as microbe-laden secretions.

Additionally, the quality of a cuff's seal against the tracheal passageway may decrease over the duration of a patient's intubation time. During this time, gases used to inflate the cuff may slowly diffuse out of the cuff through the cuff walls, which may compromise the quality of the seal. On the other hand, during surgery, anesthetic gases may enter the cuff, causing an increase in cuff pressure which may cause some discomfort for the patient. Further, coughing or other patient movements may dislodge the cuff and affect the seal against the tracheal walls.

As it is desirable to maintain a relatively constant cuff inflation pressure, often clinicians will monitor cuff inflation, typically every eight hours or so, using a device designed for this purpose. While these devices may be used to spot check cuff inflation levels, they may present certain disadvantages for the user. Generally, the use of these devices may involve additional training for healthcare providers. Additionally, the measurement of cuff inflation also may result in a certain percentage of inflation volume being lost to the measurement device itself. Thus, these devices may not be used too frequently, as they may have a negative effect on intracuff pressure.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

There is provided a medical device that includes an inflatable balloon cuff; and a gas barrier layer disposed on the balloon cuff.

There is also provided a method that includes inserting an endotracheal tube into patient's trachea, wherein the endotracheal tube comprises an inflatable balloon cuff with a gas barrier layer; and sealing the patient's trachea with the inflatable balloon cuff, wherein the inflatable balloon cuff is adapted to maintain a substantially constant intracuff pressure while in the patient's trachea.

There is also provided a method of manufacturing a medical device that includes providing an inflatable balloon cuff; and providing a gas barrier layer disposed on the balloon cuff.

There is also provided a medical device that includes an inflatable balloon cuff; and a gas barrier material embedded in the balloon cuff.

### BRIEF DESCRIPTION OF THE DRAWINGS

Advantages of the invention may become apparent upon reading the following detailed description and upon reference to the drawings in which:

FIG. 1 is a graph of gas pressure loss over time in a typical endotracheal cuff;

FIG. 2 illustrates an endotracheal tube with an inflatable balloon cuff including a gas barrier layer in accordance with aspects of the present technique;

FIG. 3 illustrates the inflatable balloon cuff of the present techniques inserted into a patient's trachea;

FIG. 4 illustrates an endotracheal tube with a multi-layer gas barrier; and

FIG. 5 is a flow chart detailing an exemplary manufacturing process for an inflatable balloon cuff including a gas barrier layer.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a medical balloon, such as an endotracheal cuff or other medical device, which may have an improved seal when inserted into a patient's trachea. In accordance with some aspects of the present technique, a medical balloon with gas barrier properties is provided that is adapted to be used with an endotracheal tube or other device. The gas barrier layer may function to maintain relatively stable intracuff pressures over time. For example, during longer intubation times, a gas barrier layer may prevent the slow egress of gases out of the cuff. Additionally, the gas barrier layer may prevent ingress of respiratory gases or anesthetic gases into the cuff. In particular, this may be advantageous during surgical procedures. Providing cuffs with relatively stable intracuff pressures may relieve the burden on healthcare workers to frequently monitor cuff pressures in order to ensure a high quality seal.

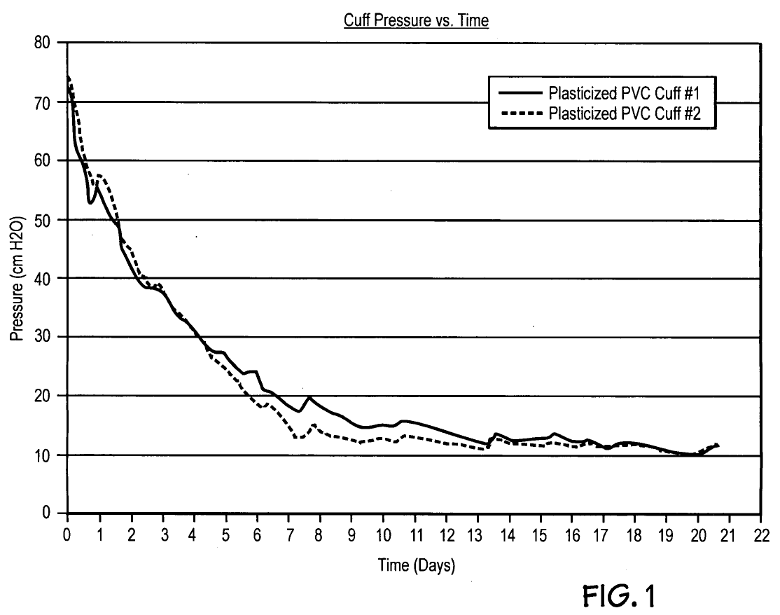

As shown in FIG. 1, over time, a typical PVC cuff may lose over half its intracuff pressure over a period of a few days. Cuffs as provided herein may have improved gas barrier properties that may retard the diffusion of gas out of the cuff. For example, a cuff as provided herein may lose less than 30%, or less than 10%, of its intracuff pressure over a period of four days. Additionally, a cuff as provided herein may lose less than 5% of its intracuff pressure over a period of 24 hours. In certain embodiments, a cuff as provided herein may lose less than 5% of its intracuff pressure over a period of eight hours. It should be understood that the reduction in loss of gas out of the cuff may depend on the particular characteristics of the gas barrier layers.

Provided herein are inflatable balloon cuffs that include a gas barrier layer. Such balloon cuffs may be used in conjunction with any suitable medical device. In certain embodiments, the cuffs, as provided herein, may be used in conjunction with a catheter, a stent, a feeding tube, an intravenous tube, an endotracheal tube, a tracheotomy tube, a circuit, an airway accessory, a connector, an adapter, a filter, a humidifier, a nebulizer, or a prosthetic.

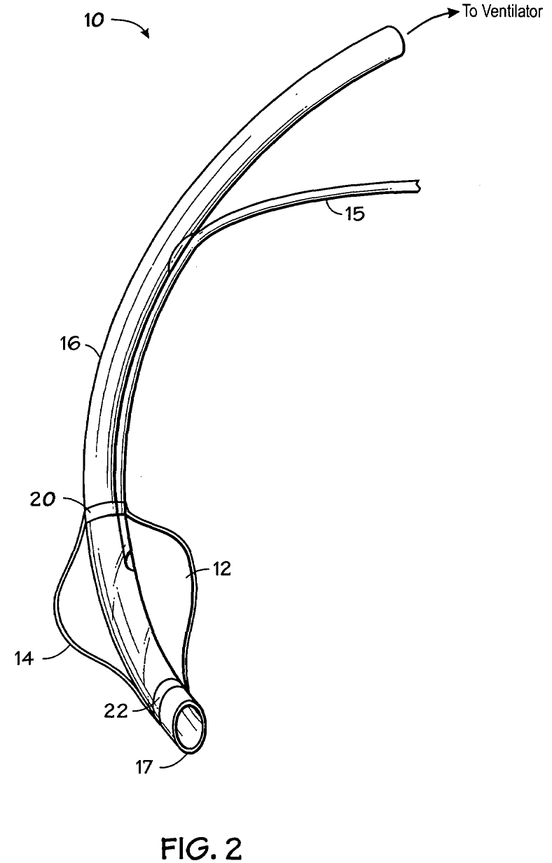

An example of an inflatable cuff used in conjunction with a medical device is an endotracheal tube 10, depicted in FIG. 2. The endotracheal tube 10 includes an inflatable cuff 12 that includes a gas barrier layer 14. The cuff 12 is disposed on a conduit 16 that is suitably sized and shaped to be inserted into a patient and allow the passage of air through the conduit 16. Typically, the cuff is disposed, adhesively or otherwise, towards the distal end 17 of the conduit 16. The cuff 12 may inflated and deflated via a lumen 15 in communication with the cuff 12, typically through a hole or a notch in the conduit 16. The cuff 12 has a proximal opening 20 and a distal opening 22 formed in the cuff walls to accommodate the conduit 16.

The cuff 12 may be formed from materials having suitable mechanical properties (such as puncture resistance, pin hole resistance, tensile strength), chemical properties (such as forming a suitable bond to the tube 16), and biocompatibility. In one embodiment, the walls of the inflatable cuff 12 are made of polyurethane having suitable mechanical and chemical properties. An example of a suitable polyurethane is Dow Pellethane® 2363-80A. In another embodiment, the walls of the inflatable cuff 12 are made of a suitable polyvinyl chloride (PVC). Suitable materials may also include polyethylene teraphthalate (PETP), low-density polyethylene (LDPE), polypropylene, silicone, neoprene, or polyisoprene. Typical cuffs 12 are formed from plasticized PVC, which is a thermoplastic polymer with an amorphous molecular structure. Depending upon the production process and the incorporated additives, various grades of PVC are obtained with sometimes differing properties. Plasticized PVC is generally somewhat permeable to water vapor.

As depicted, the barrier layer 14 may be disposed on the tissue-contacting surface of a cuff wall 11. However, it may also be suitable for the barrier layer 14 to be disposed on the opposing side of the cuff wall 11, facing the interior of the cuff, or both. Further, the barrier layer may be any suitable thickness that retains gas barrier properties while also maintaining appropriate flexibility. For example, in certain embodiments, the barrier layer 14 is less than 0.002 inches thick. In other embodiments, the barrier layer 14 is between 0.00005-0.010 inches in thickness.

The barrier layer 14 may be constructed from any suitable material. In certain embodiments, the barrier layer 14 has reduced permeability to at least one gas than plasticized PVC. For example, in certain embodiments, in may be advantageous to use metals, such as aluminum as a gas barrier layer 14. In such an embodiment, it may be advantageous to use a metal-containing barrier layer 14 on the interior surface of the cuff wall 11 instead of the tissue-contact surface in order to prevent corrosion by mucosal secretions or activation of metal allergies in a patient. In other embodiments, it may be advantageous to use a polymer or a copolymer, for example, polymers with a relatively high level of crystallization. Generally, orientation and crystallization of the polymers improves the barrier properties of the material as a result of the increased packing efficiency of the polymer chains. The particular barrier properties of polymeric materials may be determined by the chemical structure of the chain and the system morphology. Sputtered aluminum, barrier nylon, polyesters, polyvinylidene chloride (PVDC), ethylene vinyl alcohol copolymer (EVOH), polychlorotrifluoroethylene, polyethylene sealants, polyethylene terephthalate, polyethylene naphthalate (PEN), or ethylene vinyl acetate copolymer (EVA) may be appropriate gas barrier materials. Additionally, barrier films such as Surlyn® may be appropriate. Surlyn® is an ethylene and methacrylic acid copolymer with an ionically crosslinked and hydrogen bonded structure that achieves high gas barrier properties, though its chemical structure has relatively low crystallinity. Typically, a 0.001″ thick film prepared from PET has an oxygen transmission rate of about 5 cc/100 sq.in/day atm. A 0.001″ thick PET resin film may have a carbon dioxide transmission rate of about 20 cc/100 inch sq./day at 0% relative humidity. The oxygen permeability of PVDC at a thickness of 0.001″ is typically 1.2 cc/100 sq.in/day atm. PEN is similar in chemistry to PET but offers relatively better gas transmission barrier characteristics for carbon dioxide than PET. A 0.001″ thick PEN resin film has a carbon dioxide transmission rate of about 4.5 cc/100 inch sq./day at 0% relative humidity. Oxygen permeation of a 0.001″ thick film of EVOH will vary from 0.01 to 1.2 cc/100 sq.in./day atm depending on the particular grade of resin being used and the ambient conditions. A 0.001″ thick EVOH resin has a carbon dioxide transmission rate of about 0.01 to 0.2 cc/100 inch sq./day at 0% relative humidity, depending on the particular grade of the resin used.

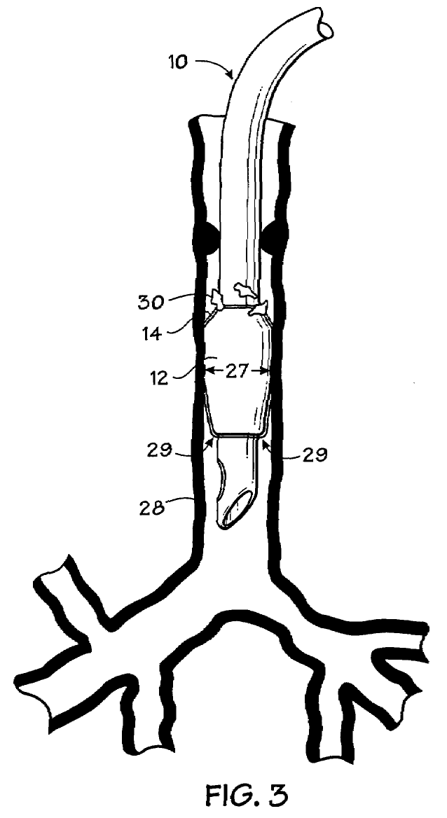

FIG. 3 shows the exemplary endotracheal tube 10 that has been inserted into a patient's trachea. The cuff 12 is inflated to form a seal against the tracheal walls 28 and may prevent secretions 30 or other detritus from passing through the trachea into the lungs. Typically, endotracheal cuffs are inflated within a patient's trachea such that the intra cuff pressure is approximately 20-30 cm H₂O. Endotracheal cuffs utilizing inflation pressures significantly greater than 25 cm H₂O, such as 100 cm H₂O, may be referred to as high-pressure cuffs, while cuffs that are designed to be inflated at pressures less than 25 cm H₂O may be considered low-pressure cuffs. It is envisioned that a barrier layer 14 may be included in both high-pressure cuffs and low-pressure cuffs. Generally, healthcare providers associate low-pressure cuffs with less discomfort for the patient. As depicted, the barrier layer 14 may slow or prevent gas from leaking out of the cuff, as depicted by arrows 27, or gas in the lungs from entering the cuff, as depicted by arrows 29. As such, the intracuff pressure may remain at the desired pressure to maintain a quality seal without causing discomfort for the patient.

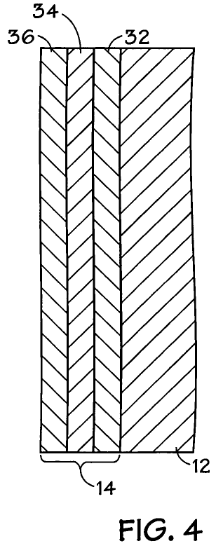

FIG. 4 illustrates an exemplary multi-component barrier layer 14. A barrier layer 14 may include multiple individual layers, depicted as 32, 34, and 36, that may be the same or different. For example, layer 36 may be a sealing layer formed from polyester or polypropylene. Layer 34 may be any suitable gas barrier material as provided herein. Layer 32 may be low density polyethylene. Alternatively, layers 32, 34, and 36 may be three different gas barrier materials, each with different gas barrier properties. For example, 32 may be PVDC, 34 may be PET, and 36 may be PEN. In another embodiment, the layers 32, 34, or 36 may have adhesion layers disposed between them. High adhesion levels can be obtained between two polyolefinic materials (polyethylene and ethylene vinyl acetate, for example) when they are extruded simultaneously through the common co-extrusion hardware as individual layers. However, in the case of polar polymers and nonpolar polymers (such as a polyolefin) the same adhesion level may not be obtained due to dissimilarities in the chemical structure of the polar and nonpolar polymers. In accordance with the instant area. A programmable parison allows the wall thickness being extruded to be controlled as a function of length. Therefore, the extruded section may have walls of varying thickness. In certain embodiments, the blow-molded cuff 12 may have a final wall thickness of 0.0002 for very thin-walled cuffs 12. Thicker walled cuffs may have wall thickness of 0.001 inches or more.

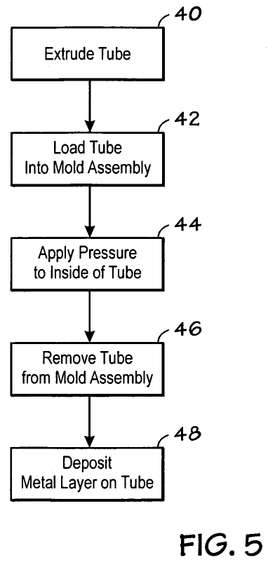

The barrier layer may be applied to the cuff 12 after it has been preformed. For example, in the flowchart of FIG. 5, a cuff may be formed from extruding a tube-shaped parison (block 40). The extruded tube, is loaded (block 42) into a blowing machine or mold assembly, such as a machine used to blow angioplasty balloons. As described above, the extruded tube may have walls of varying thickness. Balloon blowing machines typically allow process parameters such as extrusion stretch, blow pressure, and temperature to be controlled. The tube is stretched and air is blown into the tube via an air conduit, such as an air hose or nozzle, connected to a source of pressurized air, such as an air pump or pre-pressurized source, to achieve a desired positive pressure within the tube (block 44). A vacuum is applied within the tube, which now includes the blown cuff, to release the tube and cuff from the mold assembly and the tube and cuff are removed from the mold assembly (block 46).

After the cuff 12 is removed from the mold, a metal layer may be deposited on the tube, such as on the interior surface of the tube (block 48). For example, the metal layer may be an aluminum-based transparent deposition film or a silica-based transparent film. Metal vapor deposition may take place in an evacuated chamber with metal vapor emanating from a invention, polymers used for the adhesion layers may be functionalized polyolefins with the ability to create a high level permanent bond with polyolefinic resins as well as with polar resins.

A barrier layer 14 may be disposed on a cuff 12 by any suitable method, including extrusion, co-extrusion, spraying, dipping, coating, or deposition. For example, a cuff 12 as provided herein may be manufactured by a blow molding process or extrusion blow molding process. For example, the cuffs may be made by using extruded or pre-extruded tubing and applying heat and pressure appropriately within a molding cavity to achieve the desired shape (blow molding). These cuffs can also be formed by extrusion blow molding, wherein an extruder fed polymer pellets melts the polymer and feeds the molten polymer through a die to form a tube shape. This still molten polymer is then captured in a mold and air pressure is applied to expand the tube out to the walls of the mold, thus achieving the desired shape. In the extrusion blow molding process, a core or mandrel of the extruder has apertures to admit a gas such as pressurized air or an inert gas like nitrogen, into the medical device in the neighborhood of the cuff. After a length of medical device has been extruded, a mold clamps the medical device around the mandrel. As gas is admitted to the cuff area through the mandrel, the cuff expands against the mold. In the alternative, the cuff wall may be expanded in a second discrete expansion process following an extrusion or molding process, such as with a shuttle blow molding process. After initial extrusion, the extruded cuff will have a generally tubular shape with a substantially uniform wall thickness. This tubular shape may then be blown into the tapered shape. This process results in the area of the cuff with larger diameters having thinner walls because the same amount of material is stretched over a larger molten metal bath. Metal vapor may condense on the cuff 12, forming a thin layer on the surface. Multiple metal baths may be used in series to apply multiple coatings of the same or different metals. Additionally, it should be understood that the cuff may be coated with other types of metallizing processes such as, metal sputtering or electron beam metal vapor deposition. Metal may also be deposited by means of a chemical reaction such as a chemical reduction reaction.

For example, in one particular implementation, a commercially available extrusion of Dow Pellethane® 2363-80A having an inner diameter of 0.239±0.005 inches (6.0706±0.127 mm) and a wall thickness of 0.008 inches (0.2032 mm) may be blown to form a cuff 12 suitable for use with a 7.5 mm internal diameter (ID) endotracheal tube. The extruded tube may be stretched 50 to 100 mm on each end and a pressure of 1.0 to 2.0 bar is applied within the extruded tube. The extruded tube is heated for 50 to 100 seconds. As the temperature ramps up, the stretched ends of the extruded tube are relaxed to 20 to 70 mm and the air pressure within the extruded tube is increased to 1.5 to 2.1 bar. The temperature is allowed to increase to 120 to 150° C., where it is maintained for 10 to 30 seconds. The mold assembly is then cooled to 40 to 55° C., a vacuum is applied to the molded extrusion and cuff, and the extrusion and cuff are removed from the mold assembly. In another embodiment, the cuff wall thickness may be controlled by a dip coating process (not shown). For example, by controlling the withdrawal rate of a cuff mandrel from a dip coating solution, the wall thickness can be controlled. Using this control or multiple dips, it is possible to obtain even step function changes in wall thickness.

In certain embodiments, the cuff 12 may be constructed from a polymer that includes fillers with suitable gas barrier properties. For example, a polymer filler may include nanocomposites that may be incorporated into the cuff polymer prior to extrusion. An example of a suitable nanocomposite may include modified clay. Another example of a suitable filler is a plate-like fillers such as talcum powder. Plate-like fillers typically have a high aspect ratio two dimensions. The polymer filler can be incorporated by compounding it into an suitable polymer or may be incorporated as the polymeric species itself or via the monomer, which may be polymerised in situ. Gas barrier polymers can be introduced by melt blending, extrusion, or by solution blending. The polymers into which gas barrier fillers may be incorporated include nylons, polyolefins, polypropylene, polystyrene, ethylene-vinyl acetate (EVA), copolymer, epoxy resins, polyurethanes, polyimides, or polyethylene terephthalate (PET). For example, a clay nanocomposite precursor compound may be dispersed in monomer caprolactam and may be polymerised to form the nylon-6-clay hybrid as an exfoliated composite. Functionality such as hydroxyl groups can be introduced into the onium salt modifiers to improve compatibility with the nylon via hydrogen bonding as this may lead to an enhancement of desirable nanocomposite properties.

The tracheal cuffs 12 including gas barrier layers 14 of the present techniques may be incorporated into systems that facilitate positive pressure ventilation of a patient, such as a ventilator. Such systems may typically include connective tubing, a gas source, a monitor, and/or a controller. The controller may be a digital controller, a computer, an electromechanical programmable controller, or any other control system.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A medical device comprising:
an inflatable balloon cuff; and
a gas barrier layer disposed on the balloon cuff.

2. The medical device, as set forth in claim 1, wherein the gas barrier layer comprises barrier nylon, polyesters, polyvinylidene chloride, ethylene vinyl alcohol copolymer, polychlorotrifluoroethylene, polyethylene sealants, polyethylene terephthalate, polyethylene naphthalate (PEN), ethylene vinyl acetate copolymer, or any combination thereof.

3. The medical device, as set forth in claim 1, wherein the gas barrier layer comprises a film or an extrusion.

4. The medical device, as set forth in claim 1, wherein the gas barrier layer comprises a metal.

5. The medical device, as set forth in claim 4, wherein the gas barrier layer is adapted to allow no more than a 5% loss of intracuff pressure for a period of eight hours or less.

6. The medical device, as set forth in claim 1, wherein the gas barrier layer is disposed on an interior layer of the inflatable cuff.

7. The medical device, as set forth in claim 1, wherein the gas barrier layer comprises a nanocomposite.

8. The medical device, as set forth in claim 1, wherein the balloon cuff comprises polyethylene teraphthalate (PETP), low-density polyethylene (LDPE), polyvinyl chloride (PVC), silicone, neoprene, polyisoprene, or polyurethane (PU).

9. The medical device, as set forth in claim 1, comprising an endotracheal tube associated with the balloon cuff, wherein the endotracheal tube passes through a proximal opening and a distal opening of the balloon cuff.

10. The medical device, as set forth in claim 9, comprising a ventilator to which the endotracheal tube is operatively connected.

11. A method comprising:
inserting an endotracheal tube into a patient's trachea, wherein the endotracheal tube comprises an inflatable balloon cuff with a gas barrier layer; and
sealing the patient's trachea with the inflatable balloon cuff, wherein the inflatable balloon cuff is adapted to maintain a substantially constant intracuff pressure while in the patient's trachea.

12. The method, as set forth in claim 11, wherein the intracuff pressure is adapted to be at least 15 cm H₂0.

13. The method, as set forth in claim 11, wherein the intracuff pressure is adapted to be between 15-25 cm H₂0.

17. A method of manufacturing a medical device, comprising:
providing an inflatable balloon cuff; and
providing a gas barrier layer disposed on the balloon cuff.

18. The method, as set forth in claim 17, wherein the gas barrier layer comprises barrier nylon, polyesters, polyvinylidene chloride, ethylene vinyl alcohol copolymer, polychlorotrifluoroethylene, polyethylene sealants, polyethylene terephthalate, polyethylene naphthalate (PEN), ethylene vinyl acetate copolymer, or any combination thereof.

19. The method, as set forth in claim 17, wherein the gas barrier layer comprises a film or an extrusion.

20. The method, as set forth in claim 17, wherein the gas barrier layer comprises a metal.

21. The method, as set forth in claim 20, wherein the gas barrier layer comprises a vapor-deposited aluminum layer.

22. The method, as set forth in claim 17, wherein the gas barrier layer is disposed on an interior layer of the inflatable cuff.

23. The method, as set forth in claim 17, wherein the gas barrier layer comprises a nanocomposite.

24. The method, as set forth in claim 17, wherein the balloon cuff comprises polyethylene teraphthalate (PETP), low-density polyethylene (LDPE), polyvinyl chloride (PVC), silicone, neoprene, polyisoprene, or polyurethane (PU).

25. The method, as set forth in claim 17, comprising providing an endotracheal tube associated with the balloon cuff, wherein the endotracheal tube passes through a proximal opening and a distal opening of the balloon cuff.

26. The method, as set forth in claim 25, comprising providing a ventilator to which the endotracheal tube is operatively connected.

27. A medical device comprising:
an inflatable balloon cuff; and
a gas barrier material embedded in the balloon cuff.

28. The medical device, as set forth in claim 27, wherein the gas barrier material comprises a nanocomposite.

29. The medical device, as set forth in claim 27, wherein the balloon cuff comprises polyethylene teraphthalate (PETP), low-density polyethylene (LDPE), polyvinyl chloride (PVC), silicone, neoprene, polyisoprene, or polyurethane (PU).

30. The medical device, as set forth in claim 27, comprising an endotracheal tube associated with the balloon cuff, wherein the endotracheal tube passes through a proximal opening and a distal opening of the balloon cuff.

31. The medical device, as set forth in claim 30, comprising a ventilator to which the endotracheal tube is operatively connected.
